(19) United States
(12) Patent Application Publication
(10) Pub. No.: US 2010/0305610 A1
(43) Pub. Date: Dec. 2, 2010

(54) SUTURE ANCHOR

(76) Inventors: Andrew C. Kim, Temecula, CA (US); Ronald Van Elderen, Oceanside, CA (US); James W. Chang, Loma Linda, CA (US)

Correspondence Address: James W. Chang, P.O.Box 1497, Loma Linda, CA, 92354, US

(21) Appl. No.: 12/790,785

(22) Filed: May 28, 2010

Related U.S. Application Data
(60) Provisional application No. 61/182,114, filed on May 29, 2009.

(51) Int. Cl. A61B 17/04

(52) U.S. Cl. 606/232

(57) ABSTRACT

Devices and methods for locking a suture to an anchor are disclosed. In certain embodiments, a suture anchor includes a first body configured to be driven into a bone, and a second body also configured to engage the bone and coupled to the first body. At a selected embedded depth of the anchor, the second body moves towards the trailing end of the first body to facilitate a suture-lock configuration as the anchor is driven in deeper. A suture retainer such as a ring, and a flared portion at or near the trailing end of the first body, facilitate locking of a suture between the ring and either or both of the second body and the flared portion as the second body pushes on the ring that in turn pushes against the flared end. In certain embodiments, such suture-lock can be achieved substantially simultaneously as the suture anchor is driven into its final embedded depth.

### RELATED APPLICATION

This application claims priority benefit of U.S. Provisional Patent Application No. 61/182,114 filed May 29, 2009, titled “SUTURE ANCHOR,” which is incorporated herein by reference in its entirety.

### BACKGROUND

1. Field

The present disclosure generally relates to the field of medical devices, and more particularly, to devices and methods for anchoring a suture to a bone.

2. Description of the Related Art

In many surgical procedures, a need to immobilize a tissue arises. For example, a torn ligament or tendon may need to be re-attached to a bone. Such re-attachment can be achieved by using a suture to hold down the tissue to a desired location on or near the bone. The suture can be threaded through one or more locations on the tissue and be secured to one or more anchors that are embedded in the bone.

Mechanical stability of the embedded anchor is an important attribute for a suture anchor. Additionally, ease of use is another important attribute, especially in situations where surgery is performed in very limited volume—for example, in arthroscopic surgery.

### SUMMARY

In certain embodiments, the present disclosure relates to a suture anchor that includes a shaft having oppositely disposed first and second ends and an intermediate location between the first and second ends. The shaft further includes a first external thread disposed between the first end and the intermediate location and configured so as to drive the first end into a bone upon rotation of the shaft. The shaft further includes a flared portion disposed at or adjacent the second end. The shaft further includes a coupling thread disposed between the intermediate location and the flared portion. The suture anchor further includes a collar disposed between the intermediate location and the flared portion. The collar further includes a second external thread disposed on outer surface of the collar and configured so as to result in rotational movement of the collar relative to the shaft when the second external thread engages the bone after the first external thread. The collar further includes a coupling thread disposed on inner surface of the collar and configured to mate with the shaft's coupling thread to allow the rotational movement of the collar and result in longitudinal movement between first and second positions relative to the shaft. The suture anchor further includes a ring disposed between the collar and the flared portion, with the ring dimensioned so as to allow feeding of a suture between the ring and the shaft when the collar is in the first position, and so as to be pushed by the collar towards the flared portion to secure the suture between the ring and at least one of the flared portion and the collar when the collar is in the second position.

In certain embodiments, the first external thread begins at or adjacent to the first end and ends at or adjacent to the intermediate location with a first lead value. In certain embodiments, the second external thread and coupling thread of the collar are configured such that at least a portion of the second external thread begins to follow the first external thread into the bone when the collar is in the first position. In certain embodiments, the second external thread provides greater rotational resistance than that of the first external thread when engaging the bone. In certain embodiments, the greater rotational resistance of the second external thread is provided by a double-start thread having two ridges, each with a lead value that is substantially equal to the first lead value. In certain embodiments, the double-start thread is configured so that one of the two ridges begins on the outer surface so as to substantially continue from the end of the first external thread when the collar is in the first position, and the other of the two ridges begins on the outer surface offset by an amount so as to provide a new engagement with the bone. In certain embodiments, the offset amount includes a value in a range from approximately 90 degrees to 270 degrees. In certain embodiments, the offset amount is approximately 180 degrees.

In certain embodiments, the coupling thread on the shaft has the same handedness as that of the first external thread. In certain embodiments, the coupling thread on the shaft has a lead value that provides a desired amount of longitudinal movement of the collar relative to the shaft due to the rotational movement. In certain embodiments, the rotational movement is determined at least in part by the greater rotational resistance.

In certain embodiments, the lead value of the coupling thread is less than the first lead value. In certain embodiments, the lead value of the coupling thread is less than or equal to approximately ½ of the first lead value. In certain embodiments, the lead value of the coupling thread is less than or equal to approximately ¼ of the first lead value.

In certain embodiments, the lead value of the coupling thread is selected for a given second external thread configuration such that the longitudinal movement of the collar substantially coincides with the securing of the suture between the ring and the flared portion. In certain embodiments, the flared portion is positioned and configured such that the second end of the shaft is at or slightly below the surface level of the bone when the suture is secured.

In certain embodiments, the shaft defines an opening that extends longitudinally from the second end and dimensioned to receive a driver. In certain embodiments, at least a portion of the opening is defined by a torque-transfer surface dimensioned to transfer torque from the driver to the shaft for inducing the rotation of the shaft. In certain embodiments, the torque-transfer surface extends longitudinally by an amount substantially the same or close to the opening. In certain embodiments, the opening extends from the second end to a location beyond at least the intermediate location. In certain embodiments, the opening extends from the second end to the first end. In certain embodiments, the opening includes an aperture that extends through the longitudinal axis of the shaft, the aperture having a cross-sectional dimension selected to receive and transfer the torque from the driver.

In certain embodiments, the first end of the shaft is dimensioned so as to have a selected side profile. In certain embodiments, the side profile includes a taper. In certain embodiments, the tapered first end and the first external thread are configured so as to provide self-tapping capability. In certain embodiments, the selected side profile and the first external thread are configured so as to be driven into the bone via a pilot hole.

In certain embodiments, the flared portion defines the second end of the shaft so as form a countersinkable head. In certain embodiments, the countersinkable head defines a rounded circumferential edge so as to reduce likelihood of damage to the suture.

In certain embodiments, the ring includes a closed loop structure. In certain embodiments, the closed loop structure has an elliptical shape. In certain embodiments, the elliptical shape includes a substantially circular shape. In certain embodiments, the ring has a rounded cross-sectional shape so as to reduce likelihood of damage to the suture.

In certain embodiments, the shaft is formed as a single piece. In certain embodiments, the flared portion is formed after the collar and the ring are coupled to the shaft.

In certain embodiments, the shaft includes first and second pieces, with the first piece including a cylinder having the flared portion and the coupling thread, and the second piece having the first external thread and defining an opening to receive a portion of the first piece. In certain embodiments, the first piece is dimensioned to allow positioning of the ring and the collar prior to insertion of the portion of the first piece into the opening of the second piece. In certain embodiments, the first piece is press fit into the opening of the second piece so as to form the shaft.

In certain embodiments, at least one of the shaft, collar, or ring is formed from metal. In certain embodiments, at least one of the shaft, collar, or ring is formed from plastic.

In certain embodiments, the suture anchor can be packaged as a kit that includes the suture anchor and at least some instruction that facilitates use of the suture anchor.

In certain embodiments, the present disclosure relates to a method for anchoring a suture to a bone. The method includes providing first and second members coupled to each other, with the first member configured to be driven into a bone by rotation and the second member configured to follow the first member when engaging the bone at a slower rate. The coupling allows counter-rotation of the second member relative to the first member, with the counter-rotation resulting in a slower longitudinal motion of the second member relative to the first member. The method further includes providing a suture retainer configured to receive a suture prior to insertion of the first member into the bone and to secure the suture upon an amount of the counter-rotation.

In certain embodiments, the method further includes positioning the suture relative to the suture retainer, driving the first member into the bone until the second member engages the bone, manipulating the suture prior to the suture being secured, and driving the first member further until the suture is secured.

In certain embodiments, the present disclosure relates to a medical apparatus that includes a first body having proximal and distal ends along a longitudinal axis, with the proximal end and at least a portion of the first body dimensioned to receive a driver. The first body includes a first set of one or more bone-engaging features configured such that driving motion of the first body via the driver results in longitudinal motion of the first body into a bone. The apparatus further includes a second body coupled to the first body and movable between a first position adjacent the first set of one or more bone-engaging features and a second position that is closer to the proximal end of the first body. The second body includes a second set of one or more bone-engaging features configured such that when the second body is in its first position, the second set of one or more bone-engaging features engages the bone with greater resistance than the first set of one or more bone-engaging features. The coupling between the first and second bodies can be configured such that driving of the first body results in the second body moving longitudinally into the bone slower than the first body thereby resulting in the second body moving from the first position towards the second position. The apparatus further includes a suture retainer constrained between the second body and the proximal end of the first body. The retainer being can be dimensioned such that when the second body is in its first position the retainer has sufficient lateral and longitudinal play relative to the first body to allow feeding of a suture between the retainer and the first body, and when the second body is in its second position the second body pushes the retainer against the proximal end of the first body so as to secure the suture between the retainer and the first body.

In certain embodiments, the present disclosure relates to an apparatus that includes a shaft having leading and trailing ends. The shaft further includes a threaded section disposed adjacent the leading end and having a first thread disposed on outer surface of the threaded section, with the first thread having a lead value P1. The shaft further includes a coupling section disposed between the threaded section and the trailing end and having a coupling thread disposed on outer surface of the coupling section, with the coupling thread having a lead value P2. The apparatus further includes a collar having inner and outer surfaces and having a matching coupling thread disposed on the inner surface of the collar. The matching coupling thread can be configured to substantially mate with the coupling thread of the coupling section so as to allow longitudinal displacement of the collar from a first position to a second position towards the trailing end of the shaft. The lead values P1 and P2 can be selected such that a ratio of P2 and P1 is proportional to a ratio of the longitudinal displacement of the collar relative to the shaft and an embedding depth of the shaft that occurs during the longitudinal displacement.

In certain embodiments, the present disclosure relates to a suture anchor that includes an elongate first body having first and second ends. The first body further includes a bone-engaging section disposed adjacent the first end and has a first thread configured such that the bone-engaging section is capable of being driven into a bone. The first body further includes a coupling section disposed between the bone-engaging section and the second end. The suture anchor further includes a second body disposed about the coupling section of the first body. The second body is movable relative to the first body between a first position adjacent to the bone-engaging section and a second position that is closer to the second end of the first body. The second body further includes a second thread configured such that when the second body is in the first position, at least a portion of the second thread is capable of engaging the bone by following the bone-engaging section when the bone-engaging section is driven into the bone. The suture anchor further includes a suture retaining member disposed between the second body and the second end of the first body. The suture retaining member is capable of receiving a suture and configured such that when the second body moves to the second position, the suture is substantially secured relative to the suture retaining member. The suture anchor further includes a coupling mechanism formed on at least one of the first body and second body. The coupling mechanism is configured to allow movement of the second body from the first position to the second position after at least a portion of the second thread engages the bone so as to facilitate the securing of the suture relative to the suture retaining member.

In certain embodiments, the second end of the first body includes a flared portion dimensioned to constrain the suture retaining member between the flared portion and the second body. In certain embodiments, the suture retaining member includes a ring.

In certain embodiments, the second body includes an elongated collar that defines an interior surface dimensioned to substantially surround at least a portion of the coupling section. In certain embodiments, the coupling mechanism includes a coupling thread formed on at least a portion of the coupling section and a matching coupling thread formed on at least a portion of the interior surface of the elongated collar. The coupling threads can be configured to allow the second body to move towards the second end of the first body when the first body is being driven into the bone and after the engagement of the second thread with the bone. In certain embodiments, the first and second threads of the first and second bodies and the matching coupling threads can be configured such that the second end of the first body is approximately at the bone's surface when the second body reaches the second position to secure the suture.

In certain embodiments, the coupling mechanism includes a stop structure formed on an outer surface of the elongated collar. The stop structure can be configured to inhibit the elongated collar from driven further into the bone when the stop structure engages the bone's surface. In certain embodiments, the coupling mechanism further includes a coupling interface between the first body and the second body. The coupling interface can be configured to force the second body to follow the bone-engaging section into the bone when the second body is in its first position. In certain embodiments, the coupling interface is further configured so that further application of driving torque after the stop structure's engagement with the bone's surface results in the elongated collar becoming rotationally disengaged from the bone-engaging section of the first body. In certain embodiments, the coupling interface includes a cam surface defined on an end edge of the elongated collar and a substantially matching cam surface defined on an edge of the bone-engaging section. In certain embodiments, the cam surfaces are configured so as to provide a selected amount of longitudinal separation of the elongated collar followed by the rotational disengagement. In certain embodiments, the coupling section and the interior surface of the elongated collar have substantially smooth surfaces so as to facilitate both the longitudinal separation and rotational movement of the first body relative to the elongated collar as the first body is driven into the bone after the rotational disengagement. In certain embodiments, the stop structure is formed at the elongated collar's end towards the second end of the first body so as to allow the elongated collar to be substantially embedded in the bone before the rotational disengagement of the elongated collar from the bone-engaging portion of the first body. In certain embodiments, the elongated collar reaching its second position on the first body while being substantially embedded in the bone facilitates securing of the suture via engagement of the second thread of the elongated collar with the bone.

In certain embodiments, a kit can be provided, where the kit includes a suture anchor having one or more of the features summarized above, and a package for providing a desirable condition for the suture anchor. In certain embodiments, the kit can further include at least some instruction for use of the suture anchor. In certain embodiments, the kit can further include a driver configured to be capable of driving the suture anchor into a bone.

In certain embodiments, the present disclosure relates to a method for securing a suture to a bone. The method includes inserting a suture through a suture retaining ring that is part of an anchor. The anchor has a first member and a second member that is movably coupled to the first member, with each of the first and second members having at least some bone-engaging features. The ring is constrained between the first and second members and dimensioned to allow the inserting of the suture when the first and second members are in a first orientation and to secure the suture when the first and second members are in a second orientation. The method further includes attaching a driver to the anchor so as to allow turning of the anchor by providing torque to the driver. The method further includes turning the driver so as to drive the anchor into a bone such that the bone-engaging features of the first member engage with the bone. The method further includes turning the driver further to further drive the anchor such that the bone-engaging features of the second member engage with the bone. The method further includes sensing via the driver when the second member has been embedded in the bone at a selected depth. The method further includes providing an additional torque to the driver so as to induce movement of the first member relative to the second member. The method further includes continuing to turn the driver until the first and second members reach the second orientation to thereby secure the anchor.

Nothing in the foregoing summary or the following detailed description is intended to imply that any particular feature, characteristic, or component of the disclosed devices is essential.

### BRIEF DESCRIPTION OF THE DRAWINGS

These and other features will now be described with reference to the drawing summarized below. These drawings and the associated description are provided to illustrate specific embodiments, and not to limit the scope of the scope of protection.

FIG. 1A shows an example suture anchor device about to engage and be driven into a bone.

FIG. 1B shows the example anchor of FIG. 1A driven into the bone so as to secure a suture thereto in a desired manner.

FIGS. 2A-2C show longitudinal views of certain embodiments of the anchor that can allow easy positioning and maintaining of suture orientation during an anchoring process.

FIGS. 3A-3E show that in certain embodiments, the suture anchor can include first and second bone-engaging bodies coupled to allow relative longitudinal movement, such that the second body following the first body into the bone results in the second body moving into the bone at a different rate than the first body to yield the relative longitudinal movement that facilitates securing of the suture.

FIG. 4A shows a lateral view of an example embodiment of the suture anchor in an unlocked configuration and having one or more features shown in FIGS. 1-3.

FIG. 4B shows a cutaway view of the example suture anchor of FIG. 4A.

FIG. 5 shows a lateral view of the example suture anchor of FIG. 4A in a locked configuration.

FIG. 6A shows a lateral view of another example embodiment of the suture anchor in an unlocked configuration and having one or more features shown in FIGS. 1-3.

FIG. 6B shows a cutaway view of the example suture anchor of FIG. 6A.

FIGS. 7A and 7B show an example of how the suture anchor can be provided with selected thread configurations to achieve a suture lock when the anchor is embedded by a certain depth.

FIGS. 8A-8E show that in certain embodiments, a suture anchor can be configures so that the suture locking movement of the anchor's second body is initiated when the second body is substantially embedded in a bone.

FIGS. 9A-9F show a sequence of suture locking stages for an example suture anchor having the feature depicted in FIGS. 8A-8E.

FIG. 10 shows a bone-engaging portion of the first body of the suture anchor of FIGS. 9A-9F.

FIG. 11 shows an example of how the suture locking movement can be initiated by engaging surfaces of the first and second bodies of the suture anchor of FIGS. 9A-9F.

FIG. 12 shows an example design consideration that can be implemented when configuring the engaging surfaces of FIG. 11.

### DETAILED DESCRIPTION OF CERTAIN EMBODIMENTS

The present disclosure generally relates to devices and methods for securing sutures to relatively hard substrates such as bones. In many surgical procedures, a tissue may need to be attached to or be positioned relative to a bone in a secure manner. Accordingly, a suture can be threaded through such tissue and be secured to an anchor device that is or can be anchored to the bone. Depending on the circumstances, one or more of such sutures can be secured to one or more of such anchor devices.

As is appreciated by practitioners of such procedures, ease of use, robustness of anchoring mechanism and action, and residual post-surgery effect are some of the factors to be considered. As described herein, one or more features of the present disclosure can provide a number of such desirable characteristics in suture anchoring devices and methods.

It will be understood that one or more features of the present disclosure can be applied in surgical procedures in human or non-human animal subjects. Such subjects can be living or non-living subjects. In the context of living subjects, such surgical procedures can include orthopaedic surgical procedures such as arthroscopic procedures. Arthroscopic procedures are commonly performed on or about knee and shoulder joints. Such procedures can also be performed on joints associated with wrists, elbows, ankles and hips. These are some non-limiting examples of procedures where one or more features of the present disclosure can be used in an advantageous manner.

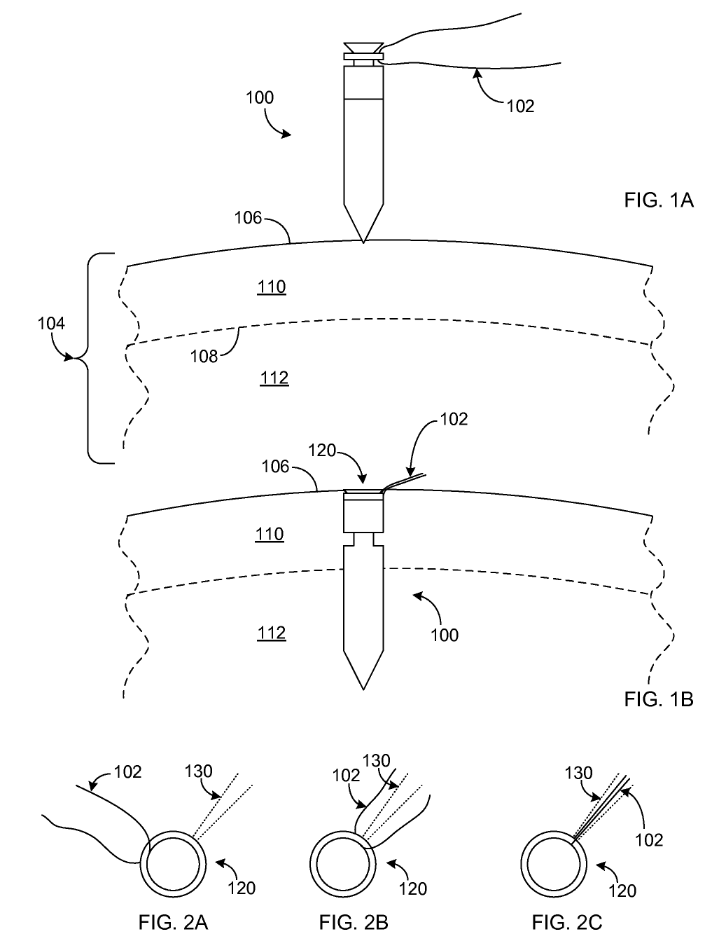

FIG. 1A depicts an example suture anchor 100 about to engage a surface 106 of a bone 104 so as to be driven into the bone 104. An example suture 102 is depicted as being threaded through a portion of the anchor 100 prior to the anchor 100 being inserted into the bone 104. In certain situations, the suture 102 can be threaded after the anchor 100 is partially inserted into the bone 104 and prior to a final locked configuration.

In certain suture anchoring situations, the bone 104 typically includes a relatively hard outer layer 110 commonly referred to as compact bone or cortical bone, and a relatively more porous inner portion 112 commonly referred to as trabecular bone or cancellous bone. For the purpose of mechanically anchoring a suture, the cortical bone 110 has mechanical properties (e.g., density and hardness) that are more desirable than that of the cancellous bone 112. Thus, as described herein, one or more features of the present disclosure can be configured to facilitate a more secure suture anchoring based on such bone properties.

FIG. 1B shows the example anchor 100 embedded in the bone 104 and the suture 102 locked in a tensioned configuration. In certain situations, it can be preferable to embed the anchor 100 so that the trailing end of the anchor 100 is positioned at or near the bone's surface 106. For example, it may not be desirable to have the trailing end of the anchor remain protruding significantly above the bone surface. It also may not be desirable to embed the anchor too deep beyond bone surface, since the unoccupied space defined between the bone surface and the trailing end of the anchor generally does not contribute to the engagement of the anchor with the cortical bone.

In certain embodiments as described herein, the anchor 100 can be configured so that driving motion that results in the anchor 100 being embedded at a desired depth in the bone 104 (e.g., trailing end substantially at the bone surface) also results in the suture 102 being locked. In certain embodiments, the suture locking motion can be substantially simultaneous with the final driving motion that results in the desired-depth embedding.

In certain embodiments as described herein, the suture anchor device can include a suture retaining mechanism that provides flexibility and ease-of-use features when retaining the suture. For example, it may be desirable to have the suture positioned and maintained along a selected azimuthal direction from the anchor (e.g., towards a sutured location on the tissue being secured). It may be further desirable to not have the suture twist and/or wrap about the anchor as the anchor is being driven into the bone.

In certain embodiments as shown in longitudinal views of FIGS. 2A-2C, a suture retaining mechanism 120 of the suture anchor can provide some or all of the foregoing features. By way of an example, suppose that the anchor is to be positioned at a given location, and that there is a desired orientation of the suture to be anchored. An example of the desired orientation of the suture is depicted as dotted lines in FIGS. 2A-2C.

FIG. 2A depicts a situation where the anchor is positioned at the given location and ready to be driven in. The suture 102 is depicted as having been threaded through the retaining mechanism 120; however, the suture 102 is not oriented properly with respect to the desired direction 130.

FIG. 2B shows that at the beginning of or during the driving process, the suture 102 can be positioned so as to be generally along the desired direction. In the example shown, the suture may remain un-tensioned during this time. In certain embodiments of the suture anchor, the suture retaining mechanism can include a ring structure that allows azimuthal freedom in suture positioning. The ring can be configured so that the suture 102 can remain at the desired orientation without twisting and/or being dragged azimuthally as the anchor is driven into the bone via, for example, rotational driving motion. In certain embodiments, the ring can be a substantially circular shaped ring. Other shaped rings are also possible.

FIG. 2C shows the suture 102 tensioned along the desired direction 130. Due to the action of the example suture retaining mechanism 120, such tensioned suture 102 can be locked in place with little or no twisting and/or wrapping.

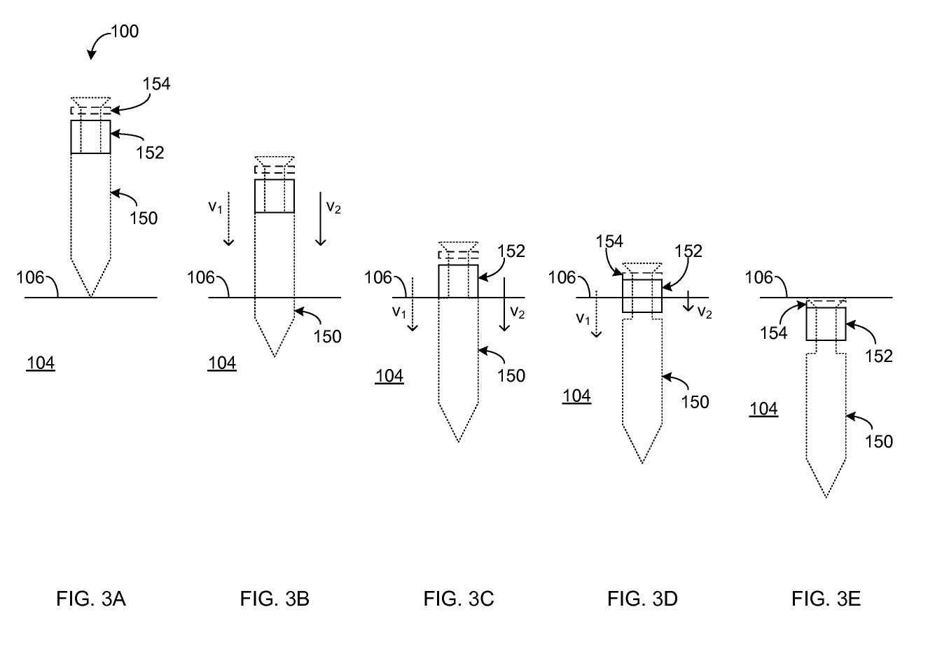

FIGS. 3A-3E show a more detailed progression of the example described herein in reference to FIGS. 1A and 1B. For the purpose of description of FIGS. 3A-3E, the suture is not shown; however, it will be understood that one or more sutures can be retained by the suture anchor as described herein.

In certain embodiments, the suture anchor 100 can include a first body 150 (depicted by dotted line) having leading and trailing ends. For the purpose of description, the leading and trailing ends (in the context of longitudinal motion during insertion into the bone) can also be referred to as distal and proximal ends (relative to a driver providing the driving motion), respectively. The first body 150 can include various features that define (going from leading end to trailing end) a bone-engaging section, a coupling section, and a suture retaining section. Various examples of such features of the sections and associated functionalities are described below in greater detail.

In certain embodiments, as shown in FIG. 3A, the suture anchor 100 can include a second body 152 (depicted by solid line) having leading and trailing ends (in the same context as the first body 150). The second body 152 can include various features that define a bone-engaging portion, a coupling portion, and a suture-retainer engaging portion. Various examples of such features of the portions and associated functionalities are described below in greater detail.

In certain embodiments, as shown in FIG. 3A, the suture anchor 100 can include a suture retainer 154 (depicted by dashed line) having leading end trailing ends (in the same context as the first body 150). Examples of various features of the suture retainer 154 and associated functionalities are described below in greater detail.

As shown in FIG. 3A, the suture anchor 100 is about to engage the surface 106 of the bone 104. More particularly, the leading end of the bone-engaging section of the first body 150 is depicted as touching the surface 106 ready to be driven into the bone 104.

In FIG. 3A, the second body 152 is depicted as being in a first position (relative to the first body 150) towards the leading end of the anchor 100 so as to provide longitudinal space for the suture retainer 154 between the trailing end of the second body 152 and the trailing end of the first body 150. Such longitudinal space can be selected to allow threading of the suture (not shown) between the suture retainer 154 and the first body 150.

In FIG. 3B, the suture anchor 100 is partially embedded into the bone 104 such that the first body 150 is engaging the bone 104 and the second body 152 is not. In certain embodiments, and as described herein in greater detail, the first and second bodies 150, 152 can be coupled so that when the second body 152 is not engaging the bone 104, its rate of longitudinal motion (indicated as v₂) relative to the bone 104 is substantially the same as that of the first body 150 (indicated as v₁). Accordingly, the second body remains substantially at its first position and the suture retention remains loose.

In FIG. 3C, the suture anchor 100 is shown to be embedded even deeper into the bone 104 such that the second body 152 begins to engage the bone 104. Until such engagement is made, v₂ remains substantially the same as v₁. Accordingly, the second body remains substantially at its first position and the suture retention remains loose.

In FIG. 3D, the suture anchor 100 is shown to be embedded even deeper into the bone 104 such that the second body 152 is engaging the bone 104. In certain embodiments, the second body 152 can be configured so that its engagement with the bone 104 results in its rate of longitudinal motion relative to the bone 104 (v₂) being different than that of the first body 150 (v₁). In certain embodiments, such difference between v₂ and v₁ can result from v₂ becoming smaller as the second body's longitudinal motion slows down due to the second body's engagement with the bone 104.

There are a number of ways of inducing slower longitudinal motion of the second body 152 when it engages the bone 104. By way of a non-limiting example, the second body 152 can be configured to provide greater resistance in its engagement with the bone 104 than that of the first body 150. There are a number of ways of providing such resistance; and some non-limiting examples are described below in greater detail.

As shown in FIG. 3D, the reduction of v₂ results in the second body 152 moving away from its first position (relative to the first body 150) towards the trailing end of the first body 150. On its way, the second body 152 is depicted as engaging the suture retainer 154; and further movement of the second body 152 results in the suture retainer 154 being pushed towards the trailing end of the first body 150.

In FIG. 3E, the suture anchor 100 is shown to be embedded into a final depth where the trailing end of the first body 150 is at or near the surface 106 of the bone 104. As described herein, the final depth does not necessarily need to result in such a flush embedding. Some final depths can include situations where the trailing end of the suture anchor 100 protrudes above or sunk below the surface 106 by some amount.

As shown in FIG. 3E, the second body 152 is depicted as having pushed the suture retainer 154 towards the trailing end of the first body 150 so as to lock the suture retainer 154 tightly between the second body 152 and the end portion of the first body 150. In such a configuration, the suture can be locked from movement away from the anchor 100. Accordingly, in certain situations, the suture can be tensioned prior to such locking so as to provide an effective anchoring functionality.

In certain embodiments, the suture anchor 100 can be configured so that the longitudinal motion of the second body 152 (relative to and towards the trailing end of the first body 150) from its engagement with the bone 104 (e.g., at FIG. 3C) to suture lock (e.g., at FIG. 3E) is substantially synchronized to yield the desired embedded depth (such as that of FIG. 3E) as the suture lock is achieved. Examples of first and second bodies and couplings that can facilitate such synchronization are described below in greater detail.

The foregoing feature can be particularly useful when a user driving the suture anchor 100 is able to detect an initial or other bone-engagement of the second body 152. In certain embodiments, resistance of the second body's bone-engagement can be detected by tactile feedback from the anchor 100 through a driver. Thus, with such a capability, the user can be aware that suture locking motion has begun so as to facilitate final suture configuration (e.g., tensioning of the suture) prior to suture lock.

In certain embodiments, the suture lock position can also be detected by the user. For example, the suture retainer 154 and/or the trailing end of the first body 150 can be configured to provide detectable difference(s) in their/its engagement with the bone as the anchor 100 attempts to be driven in further. Examples of suture retainer and trailing end that can provide such detectable differences are described below in greater detail.

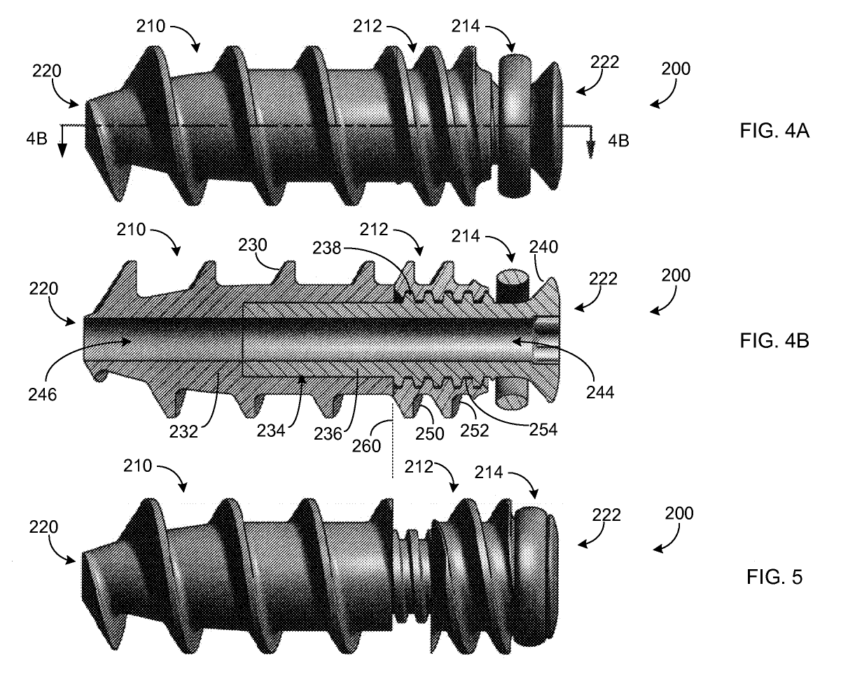

FIGS. 4-6 show non-limiting example configurations of the suture anchor having one or more of the features described in reference to FIGS. 1-3. The examples described in reference to FIGS. 4-6 have various screw threads that facilitate driving motions and coupling of the first and second bodies. It will be understood that bone-engaging features of the first and/or second bodies can include features other than screw threads without departing from one or more concepts described herein. Similarly, coupling features that couple the second body to the coupling section of the first body can include features other than screw threads without departing from one or more concepts described herein.

In certain embodiments, various parts of the suture anchor can be formed from materials such as metals and/or plastics. Preferably, such materials have properties such as biocompatibility and suitable for surgical implantation. Some non-limiting examples of materials that can be used to fabricate the suture anchor include: stainless steel, titanium, cobalt-chrome, plastic, and biocompatible polymers such as PEEK-based products.

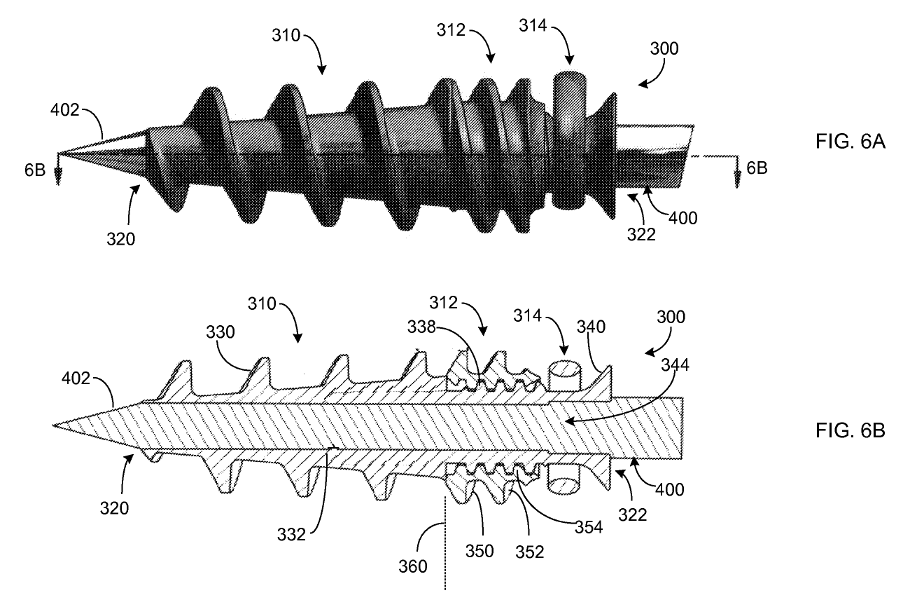

In certain embodiments, the first body (150 in FIGS. 3A-3E) can be formed as a single piece such as the example shown in FIGS. 6A and 6B, or as an assembly of two or more pieces such as the example shown in FIGS. 4A, 4B, and 5. Such different configurations can facilitate or be dictated by, for example, different fabrication processes for the anchor suture.

FIG. 4A shows a lateral view of an example suture anchor 200 having a first body 210, a second body 212, and a suture retainer 214. In the example shown, leading and trailing ends (220, 222) of the first body 210 also define the leading and trailing ends of the anchor 200.

In FIG. 4A, the second body 212 is in its first position relative to the first body, such that the suture retainer 214 is in an unlocked configuration to allow threading and movement of a suture (not shown). As shown, FIG. 4B depicts a lateral cutaway view of the suture anchor 200 of FIG. 4A.

As shown in FIG. 4B, the example first body 210 includes a first piece 236 and a second piece 232 joined together so as to form a shaft shaped first body 210. In the example shaft 210 shown, the second piece 232 defines a cylindrical shaped recess 234 that extends longitudinally from its trailing end and dimensioned to receive the leading end of the first piece 236. In certain embodiments, such first and second pieces 236, 232 can be press fit so as to allow the two pieces to behave as a single piece during use.

In the example anchor 200, the second piece 232 forms the bone-engaging section, and a longitudinal portion of the first piece 236 forms the coupling section, of the anchor as described herein in reference to FIGS. 3A-3E. In certain embodiments, the bone-engaging section can have one or more threads configured to engage the bone and be driven in longitudinally in response to an applied torque.

In the example anchor 200, the bone-engaging section 232 is depicted as having a thread 230 that extends substantially from the leading end 220 to the trailing end of the bone-engaging section 232. In FIG. 4B, the longitudinal location where the thread 230 ends is depicted as an intermediate location 260.

In certain embodiments, the coupling section can include one or more features that allow at least some rotational motion between the first and second bodies (210, 212). Such one or more features can also provide a functionality where the first body 210 provides at least some longitudinal pulling of the second body 212 as the first body 210 is driven into the bone. In certain embodiments, such functionalities of the coupling section can be provided by a coupling thread that couples with a matching thread on the second body 212.

In the example anchor 200, the coupling section on the first piece 236 is depicted as having a coupling thread 238 that extends substantially from the intermediate location 260 towards the trailing end 222 by an amount that allows the suture retainer 214 to be in an unlocked configuration. To mate with the example coupling thread 238, the second body 212 can be a hollow cylindrical collar having an outer surface and an inner surface. The inner surface can include a matching coupling thread 254 that extends longitudinally between the leading and trailing ends of the second body 212.

In the example anchor 200, outer surface of the second body 212 (e.g., a collar) can define the bone-engaging section described herein in reference to FIGS. 3A-3E. In certain embodiments, such bone-engaging section can include one or more threads configured to engage the bone.

In the particular example of the anchor 200 shown in FIGS. 4A-4C, the bone-engaging section includes first and second threads (250, 252) that extend substantially the entire longitudinal length of the second body 212. The first thread 250 can be configured so that when the second body 212 is in an unlocked position as shown in FIG. 4B, the first thread 250 substantially continues from the end of the bone-engaging thread 230 of the first body 210. In certain embodiments, the first thread 250 has a lead value that is substantially the same as that of the first body's bone-engaging thread 230. Further, the first thread 250 can have a profile that is substantially the same as that of the first body's bone-engaging thread 230.

The second thread 252 can be configured so as to lag behind the first thread 250. Such lagging can be by an amount that is greater than about 0 degree and less than about 360 degrees. Preferably, the lagging amount is in a range that is between about 90 and 270 degrees. For the example anchor 200, the lagging amount is approximately 180 degrees.

In certain embodiments, the second thread 252 can begin at or near the leading end of the second body 212, and the beginning portion of the second thread 252 can be ramped so as to allow the second thread to cut a new groove in the bone. Such cutting and engagement with the new groove in the bone can provide an increased rotational resistance of the second body 212 to thereby provide the resistance described herein in reference to FIGS. 3A-3E. It will be understood, however, that there are a number of ways to achieve such difference in bone-engaging resistances between the first and second bodies (150, 152 in FIGS. 3A-3E, and 210, 212 in FIGS. 4A and 4B).

For example, instead of dual threads (250, 252), a single thread can be provided on the second body 212 and be configured to substantially continue from the end of the first body's bone-engaging thread 230. The beginning portion of such a single thread can have a profile that is similar or substantially the same as that of the ending portion of the first body's thread 230. The single thread can then gradually change its profile so as to provide greater rotational resistance against the bone. For example, the thread profile can be gradually broadened to provide the additional resistance.

In certain embodiments, even a difference in surface textures of the first and second bodies may be able to provide the difference in bone-engaging resistance. For example, suppose that the first body's thread 230 and the first thread 250 of the second body 212 are substantially identical in profile and have substantially the same lead value. Then, a smooth surface on the thread 230 and a rougher surface on the thread 250 may provide sufficient difference in rotational resistance when the thread 230 of the second body engages the bone.

As described in reference to FIGS. 3A-3E, the second body 212 follows the first body 210 into the bone. The difference in the bone-engaging resistance (in this example, a greater rotational resistance) between the first and second bodies 210, 212 results in the second body 212 moving into the bone slower than that of the first body 210. Such slower movement of the second body 212 can result in the second body 212 being at a second position where it has pushed the suture retainer 214 into a locked configuration to secure the suture (not shown).

FIG. 5 shows the example anchor 200 where the second body 212 is in the second position to provide the suture locking. To provide such a locking configuration, the second body 212 is shown to have moved a certain amount longitudinally towards the trailing end 222 of the first body 210. Examples of configuring the bone-engaging threads and the coupling thread to substantially synchronize the backward movement of the second body 212 (relative to the first body 210) with the desired embedding depth of the anchor 200 are described below in greater detail.

In certain embodiments as shown in FIGS. 4A, 4B, and 5, the suture retainer 214 can include a ring structure. Such a ring structure can be a closed, and such a closed ring can provide suture locking along substantially all azimuthal directions about suture anchor 200. However, fully closed ring structure is not a requirement for the purpose of locking the suture via the relative motion of the second body 212. For example, a partially open ring can be constrained near the trailing edge 222 of the anchor 222 and provide locking within an azimuthal range less than 360 degrees.

In certain embodiments, the ring structure 214 can be substantially circular. Such a circular shaped retainer can provide azimuthal symmetry in suture locking direction. However, the circular shape is not a requirement. For example, a full internal symmetry may not be desired in some situations. A shape such as an ellipse can be selected to limit such internal symmetry. In such situations, full azimuthal locking coverage can still be achieved. For example, an elliptical ring and a corresponding trailing end with an elliptical cross-section can be dimensioned to provide appropriate mating so as to lock the suture along any azimuthal direction.

Preferably, the ring structure 214 has a rounded cross-sectional shape to reduce likelihood of damage to the suture. Similarly, portions of the first and second bodies 210, 212 that come into contact (or likely to come into contact) with the suture can be shaped appropriately (e.g., smoothly) to reduce likelihood of damage to the suture. For example, portions of the coupling threads (238, 254) and the bone-engaging threads (250, 252) proximate the ring 214 can be removed, rounded, or dulled.

In the example anchor 200, the suture retaining ring 214 can be constrained between the second body 212 and a flared portion 240. To achieve such constraint, the inner diameter of the ring 214 (assuming a circular ring) can be made to be greater than the unthreaded portion (between the coupling thread 238 and the flared portion 240) but less than the largest diameter of the flared portion 240. Similarly, the inner diameter of the ring 214 can be less than the major diameter of the outer portion of the second body 212.

In such an example configuration, locking of the suture can be achieved by the suture being squeezed between the ring 214 and the flared portion 240, and/or between the ring 214 and the trailing end of the second body 212.

In the example two-piece first body 210 shown in FIG. 4B, the inner diameter of the ring 214 can be made to be greater than the major diameter of the first piece 236. Such a configuration can facilitate an assembly process where the ring is slid over the coupling thread 238 of the first piece 236 prior to installation of the second body 212 (to the first piece 236) and press fitting of the first piece 236 into the second piece 232.

As shown in FIG. 4B, the example flared portion 240 is depicted as being substantially at the trailing end 222 of the first body 210. Such a position is not a requirement for the purpose of locking the suture via the relative motion of the second body 212. For example, the trailing end of the first body 210 may extend beyond the flared portion 240.

In the example anchor 200, the first and second pieces (236, 232) of the first body 210 are depicted as defining respective apertures (244, 246) that extend longitudinally. The apertures (244, 246) can be dimensioned to receive a driver (not shown), and at least some portions of the apertures (244, 246) can be configured to allow transfer of the driver's torque. While it is not necessary to have the driver-engaging recess extend all the way through the anchor, there are situations where such a configuration can be desirable. Such design considerations are described below in greater detail.

FIGS. 6A and 6B show another non-limiting example of a suture anchor 300 where the first body is formed as a single piece. Various features and functionalities of the anchor 300 are similar to those of the anchor 200 described in reference to FIGS. 4A and 4B. More particularly, features and functionalities (not related to the single-piece/two-piece difference) associated with first and second bodies (310, 312), a suture retainer 314, leading and trailing ends (320, 322), bone-engaging threads (330, 350, 352), coupling threads (338, 354), and a flared portion 340 are generally similar to the first and second bodies (210, 212), the suture retainer 214, the leading and trailing ends (220, 222), the bone-engaging threads (230, 250, 252), the coupling threads (238, 254), and the flared portion 240 described in reference to FIGS. 4A and 4B.

As shown in FIG. 6B, the first body 330 is depicted as being formed by a single piece 332. As such, an assembly process for the anchor 300 can include the flared portion 340 being formed after installation of the second body 312 and the ring 314 from the trailing end 322.

As shown in FIGS. 6A and 6B, the example profile of the first body 310 shows a gradual taper from the intermediate location 360 to the leading end 320, whereas the profile for the first body 210 of the example anchor 200 shows more of a straight shaft with a rounded tip. In various embodiments, different profiles of the suture anchor are possible. Some design considerations concerning the profiles are described below in greater detail.

In FIG. 6B, the example first body 310 is shown to define a driver-receiving opening 344 that extends all the way through to the leading end 320. Similar to the apertures 244 and 246 of FIG. 4B, the aperture 344 can be dimensioned to receive a driver 402, and at least some portion of the aperture 344 can be configured to allow transfer of the driver's torque.

Similar to the example suture anchor shown in FIG. 4B, it is not necessary to have the driver-engaging recess 344 extend all the way through the anchor 300. In certain embodiments, a driver-engaging recess can be relatively shallow, such as that found on some screw heads.

In certain embodiments, the suture anchor does not necessarily need a recess to engage a driver. For example, a socket tipped driver can drive the anchor's trailing end dimensioned to fit into the socket.

In embodiments where the driver is engaged by a recess (such as in the example anchors 200 and 300), a deeper recess can provide more distribution of torque engaging surface to reduce the likelihood of damage to the driver and/or the anchor. For example, in the example configuration of the first body 200 in FIG. 4B where the first piece 236 can be press fit into the second piece 232, suppose that the opening 244 does not extend into the second piece 232 such that a driver provides torque only to the first piece 236. With the second piece 232 engaging the bone and the first piece 236 being driven, there may be sufficient shear force therebetween to separate the two pieces. If the driver-receiving opening extends into the second piece 232, however, such a problem can be avoided.

In certain situations, factors such as anchor dimension, anchor material, and/or driver profile can contribute to determining the extent of the driver-engaging depth. For example, materials such as plastic can have mechanical properties (e.g., softer) that make them more susceptible to deformation under torque. Thus, anchors having such materials can benefit from a driver-engaging opening that extends a greater length.

In various embodiments of the present disclosure, the driver-engaging opening (such as 244 in FIGS. 4B and 344 in FIG. 6B) can be dimensioned to receive and engage various driver profiles. Such driver profiles can include, but are not limited to, Phillips, Robertson (square), hex, torx, and high-torque capable profiles such as Motorq Super.

In certain embodiments, the suture anchor's dimensions and profiles can be dictated or influenced by the materials used. In anchors that are formed from relatively soft plastics, it may be preferable to have the driver-engaging opening extend throughout the anchor (as described above), and to provide sufficient wall thickness between the driver-engaging opening and the outer surface of the first body. Such example requirements can lead to, for example, a straight-walled profile that does not have a taper near the leading end. For such an example anchor profile, a driver having a pointed tip (such as the example driver 402 in FIGS. 6A and 6B) can guide the anchor into the bone via, for example, an existing pilot hole.

In certain embodiments, the suture anchor and/or the bone-engaging threads on the first body can be configured to be driven into the bone via such a pilot hole, via self-tapping features formed at or near the leading end, or any combination therebetween.

In certain embodiments, various configurations of bone-engaging threads can be implemented to accommodate different applications and/or different bone properties. In the example second bodies (212 in FIGS. 4B and 312 in FIG. 6B), dual threads are provided as an example of introducing additional rotational bone-engagement resistance. In certain situations, such additional thread(s) can provide improved anchoring properties in denser bones such as the cortical bone. As such, a selected portion of the first body (210 in FIGS. 4B and 310 in FIG. 6B) can be provided with additional features such as an additional thread. For example, a second thread can be provided on the first body; and to maintain sufficient difference in bone-engaging resistances between the first and second bodies, the second thread can have a lower profile.

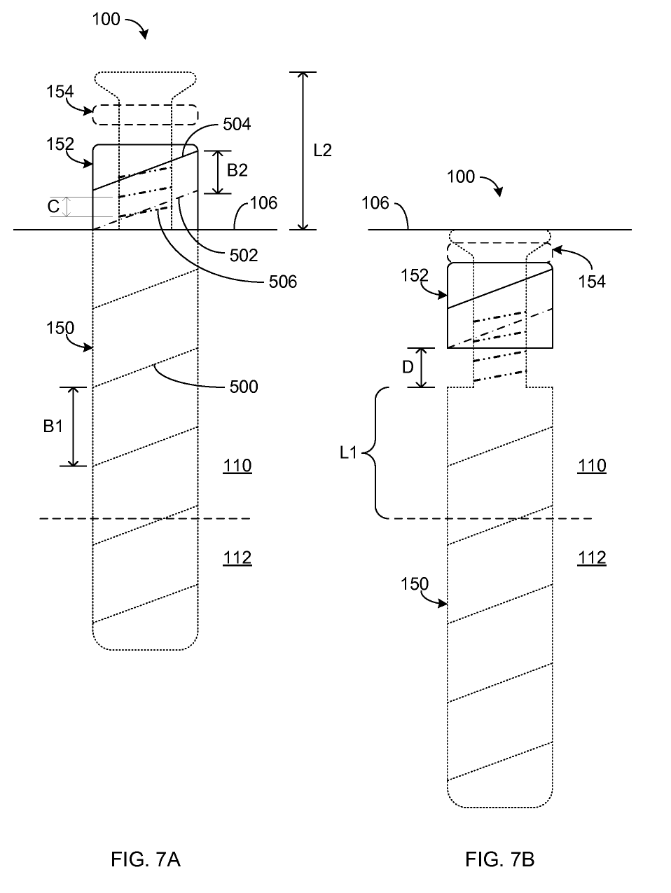

As described herein, certain embodiments of the suture anchor can be provided with bone-engaging threads and coupling threads (for coupling the first and second bodies) to facilitate the suture locking motion as the anchor is being driven into the bone. In such configurations, the bone-engaging threads and the coupling threads can be selected so that the suture lock is achieved when the anchor is embedded in the bone by a desired depth. FIGS. 7A and 7B show an example of how such embedding-locking synchronization can be achieved.

FIG. 7A shows an example of the suture anchor 100 in a partially-embedded position (similar to that in FIG. 3C) where the second body 152 begins to engage the surface 106 of the bone. More particularly, the second body 152 begins to engage the cortical bone 110. At such a position, the anchor 100 protrudes above the surface 106 by an amount indicated as “L2,” and the anchor 100 remains in an unlocked configuration.

FIG. 7B shows the example anchor 100 embedded into the bone by a desired amount. More particularly, the anchor 100 is shown to have been further embedded longitudinally (from the position of FIG. 7A) by approximately L2. During such longitudinal motion of the anchor 100 by L2, the second body 152 is shown to have moved towards the anchor's trailing end by an amount indicated as “D” so as to provide the locking pressure to the suture retainer 154.

To achieve the locking motion of the second body 152 by approximately D during the longitudinal motion of the anchor 100 by approximately L2, one can provide selected lead values for the bone-engaging threads of the first and second bodies (150, 152) and the coupling threads (indicated as 506 in FIG. 7A) between the first and second bodies (150, 152).

The bone-engaging thread (indicated as 500 in FIG. 7A) of the first body 150 generally determines the rate of longitudinal motion of the anchor 100 as a whole; thus, the thread 500 can be provided with a lead value indicated as “B1.” To longitudinally move the anchor 100 by an amount L2, the first body 150 needs to be rotated by L2/B1 turns. For the purpose of description, the fraction L2/B1 can be referred to as N_{final}, with an understanding that N_{final} may or may not be an integer.

During the final driving rotation by N_{final} turns, second body 152 is shown to have backed-up towards the first body's trailing end by an amount D. To accommodate such motion of the second body 152 relative to the first body 150 during N_{final} turns, the coupling threads between the two bodies (150, 152) can be provided with a lead value (indicated as “C” in FIG. 7A) of D/N_{final}. Thus, C=D/(L2/B1). Rearranging the terms, ratio of the lead value C of the coupling thread 506 and the lead value B1 of the first body's bone-engaging thread 500 can be expressed as:

C/B1=D/L2. (Eq. 1)

For the example anchors described herein in reference to FIGS. 4-6, the ratio of D/L2 for both anchors (200, 300) is approximately ¼. Thus, lead values C and B1 for the coupling threads and the bone-engaging threads, respectively, can be selected such that their ratio is also approximately ¼. For example, if B1 is approximately 2.5 mm, C can be approximately 0.625 mm.

In certain embodiments, the “backward” motion of the second body 152 (relative to the first body 150) can begin when the second body 152 first engages the bone surface 106. As described herein, a rotational resistance encountered by the second body 152 against the bone can induce such a relative motion of the second body 152. Such rotational resistance of the second body 152 can be due to one or more additional features such as the second bone-engaging thread 504. Even without such additional features, there may be sufficient rotational resistance of the second body 152 (which may or may not be greater than that of the first body 150 per unit longitudinal length) to induce the backward relative motion of the second body 152.

In certain embodiments, the thread-configuration parameter of Equation 1 can be used as a basis for a design of the anchor suture. There may be effects that can contribute to deviation of the final driving motion being synchronized with the desired locking motion. Thus, the initial design may be refined based on, for example, empirical data so as to achieve the desired synchronization.

As further shown in FIGS. 7A and 7B, various dimensions of the suture anchor 100 can be selected so that when the anchor 100 is in its embedded position, at least a portion of the first body's (150) bone-engaging thread 500 remains in engagement with the cortical bone 110. In FIG. 7B, a portion of the first body 150 indicated by a length “L1” is depicted as engaging the cortical bone 110. Further, and as described herein, the second body's (152) bone-engaging thread(s) (502, 504) can engage the cortical bone 110 as well. The combined cortical-bone engagements by the first and second bodies (150, 152) can provide a secure embedding of the anchor 100, and a secure locking of the suture thereto.

In the various examples described in reference to FIGS. 3-7, it is generally assumed that the second body's (152) longitudinal separation from the first body's (150) bone-engaging thread portion is induced by the second body's (152) initial engagement with the surface of the cortical bone. In certain embodiments, however, a suture anchor can be configured such that the longitudinal separation between the second body 152 and the first body's (150) bone-engaging thread portion occurs after at least a portion of the second body 152 has engaged the cortical bone.

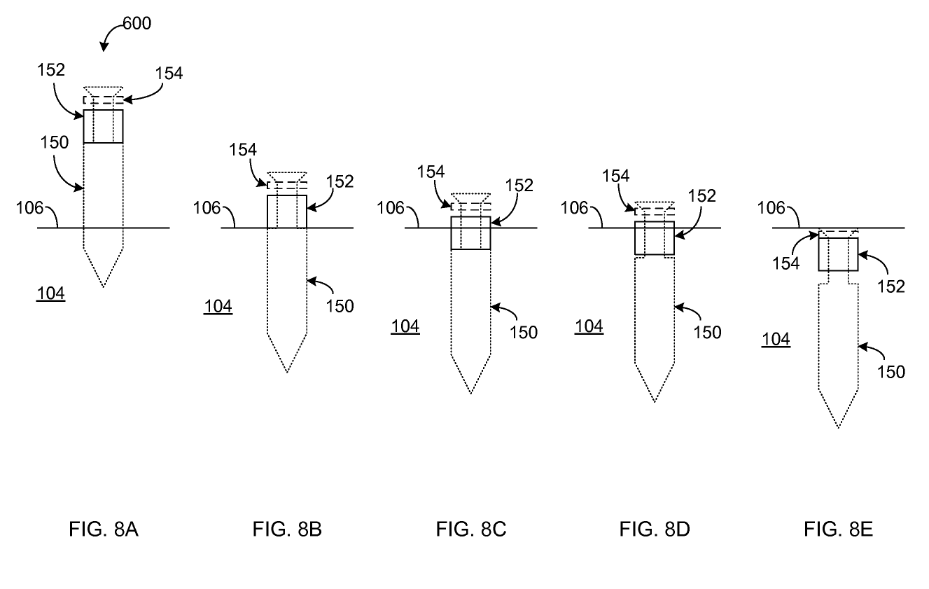

FIGS. 8A-8E show an example progression of a suture anchor 600 as it is inserted into a bone 104. Similar to the example described in reference to FIGS. 3A-3E, a suture is not shown; however, it will be understood that one or more sutures can be retained by the suture anchor as described herein.

In certain embodiments, a suture anchor 600 can include a first body 150 (depicted by dotted line) having leading and trailing ends. For the purpose of description, the leading and trailing ends (in the context of longitudinal motion during insertion into the bone) can also be referred to as distal and proximal ends (relative to a driver providing the driving motion), respectively. The first body 150 can include various features that define (going from leading end to trailing end) a bone-engaging section, a coupling section, and a suture retaining section.

In certain embodiments, as shown in FIG. 8A, the suture anchor 600 can include a second body 152 (depicted by solid line) having leading and trailing ends (in the same context as the first body 150). The second body 152 can include various features that define a bone-engaging portion, a coupling portion, and a suture-retainer engaging portion. In certain embodiments, the second body 152 can also include a feature that facilitates longitudinal separation of the second body 152 from the bone-engaging section of the first body 150 after the second body has been inserted at least partially into the bone 104. A non-limiting example of such a feature is described below in greater detail.

In certain embodiments, as shown in FIG. 8A, the suture anchor 600 can include a suture retainer 154 (depicted by dashed line) having leading and trailing ends (in the same context as the first body 150). Examples of various features of the suture retainer 154 and associated functionalities are described herein in greater detail.

As shown in FIG. 8A, the suture anchor 600 is about to engage the surface 106 of the bone 104. More particularly, the leading end of the bone-engaging section of the first body 150 is depicted as touching the surface 106 ready to be driven into the bone 104.

In FIG. 8A, the second body 152 is depicted as being in a first position (relative to the first body 150) towards the leading end of the anchor 600 so as to provide longitudinal space for the suture retainer 154 between the trailing end of the second body 152 and the trailing end of the first body 150. Such longitudinal space can be selected to allow threading of the suture (not shown) between the suture retainer 154 and the first body 150.

In FIG. 8B, the suture anchor 600 is shown to be embedded into the bone 104 such that the second body 152 begins to engage the bone 104. At this stage, the second body remains substantially at its first position and the suture retention remains loose.

In FIG. 8C, the suture anchor 600 is shown to be embedded even deeper into the bone 104 such that the second body 152 is engaging the bone 104. In certain embodiments, the second body can remain substantially at its first position such that the suture retention remains loose.

In FIG. 8D, the suture anchor 600 is shown to be driven into the bone deeper, and the second body 152 is shown to have begun its separation from the bone-engaging section of the first body 150. Accordingly, the distance between the trailing end of the second body 152 and the trailing end of the first body 150 begins to decrease. As the second body 152 moves relative to the first body 150 away from its first position, the second body engages the suture retainer; and further movement of the second body 152 results in the suture retainer 154 being pushed towards the trailing end of the first body 150.

In FIG. 8E, the suture anchor 600 is shown to be embedded into a final depth where the trailing end of the first body 150 is at or near the surface 106 of the bone 104. As described herein, the final depth does not necessarily need to result in such a flush embedding. Some final depths can include situations where the trailing end of the suture anchor 600 protrudes above or sunk below the surface 106 by some amount.

As shown in FIG. 8E, the second body 152 is depicted as having pushed the suture retainer 154 towards the trailing end of the first body 150 so as to lock the suture retainer 154 tightly between the second body 152 and the end portion of the first body 150. In such a configuration, the suture can be locked from movement away from the anchor 600.

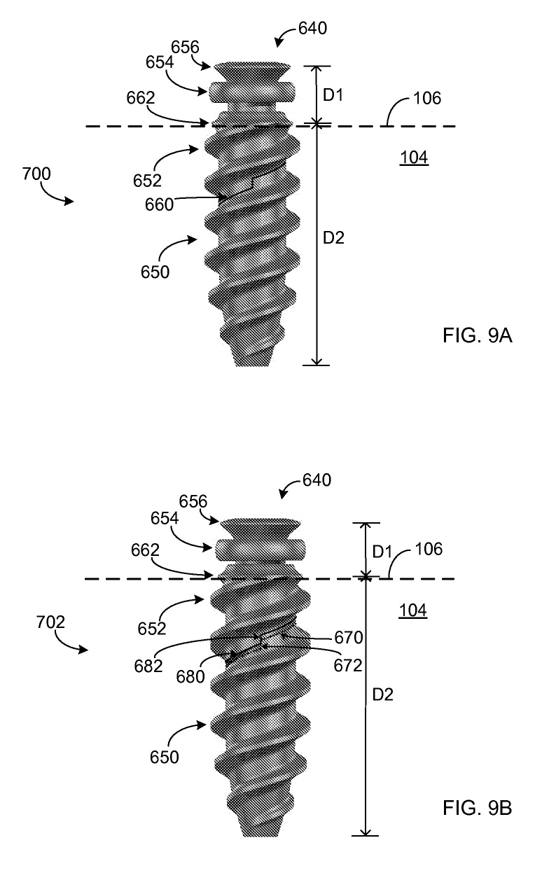

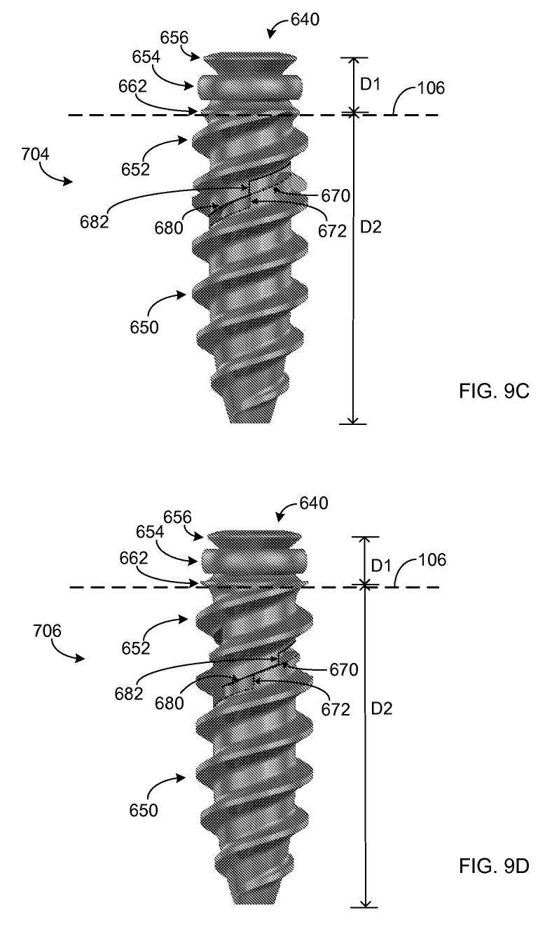

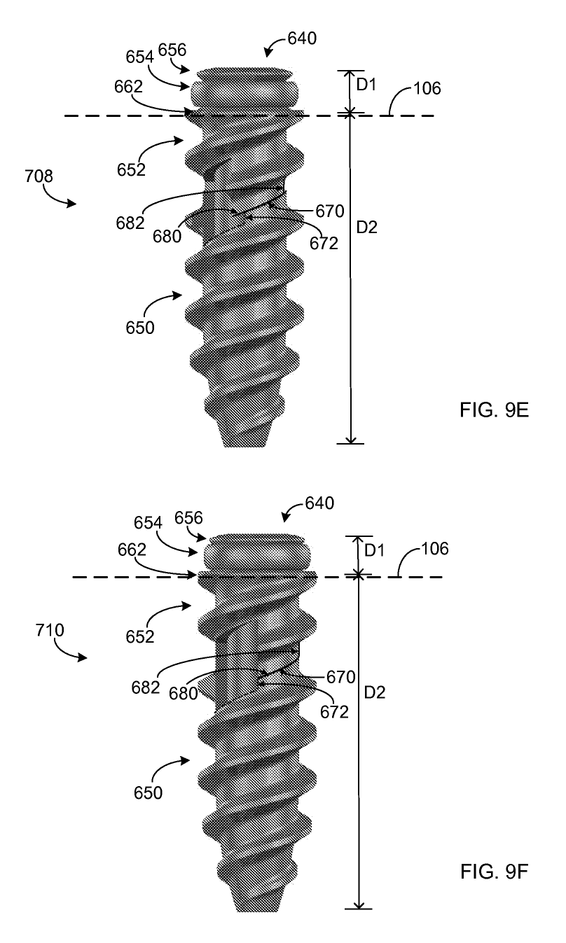

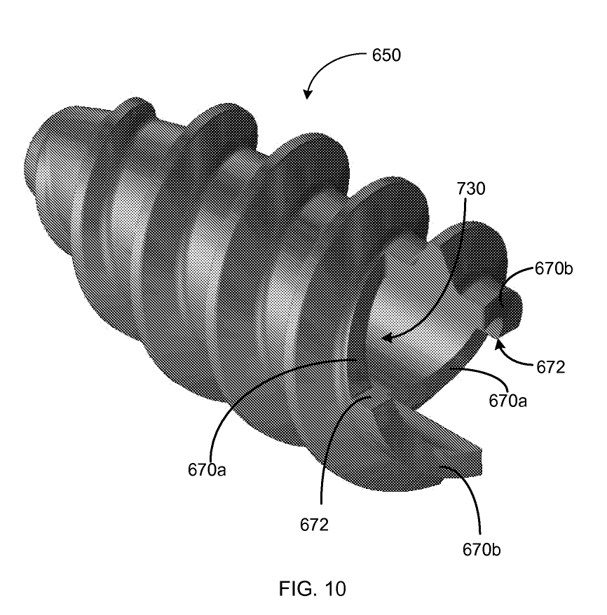

FIGS. 9A-9F show a sequence of suture locking achieved by an example suture anchor 640 that is configured to have its second body (152 in FIGS. 8A-8E) begin its separation after the second body has engaged the bone (104). For the purpose of description of the example sequence, the suture anchor 640 in FIG. 9A is assumed to have been driven into the bone such that the anchor 640 is in a stage similar to that depicted in FIG. 8C. Also, FIG. 10 depicts in greater detail a portion of the anchor 640 that facilitates coupling of the first and second bodies of the anchor 640.

As shown in FIGS. 9A-9F, the example suture anchor 640 is depicted as having a first body 650 coupled to a second body 652. The suture anchor 640 is further shown to include a suture retaining ring 654 that can be constrained between the second body 652 and a flared portion 656 at or near the first body's (650) trailing end. In certain embodiments, the suture retaining ring 654 and the flared portion 656 can be similar to those described herein in reference to FIGS. 4-6.

As shown in FIGS. 9A-9F and 10, the first body 650 can include a bone-engaging thread pattern. In certain embodiments, such a thread pattern can be similar to those described herein in reference to FIGS. 4-6.

In certain embodiments, the first body 650 can include two separate pieces that can be joined so as to form a shaft shape for the first body, in a manner similar to the example described in reference to FIG. 4B. Thus, FIG. 10 shows an example cylindrical shaped recess 730 that can be defined by the first body 650, in a manner similar to the example recess 234 described in reference to FIG. 4B.

In certain embodiments, the suture anchor 640 can be configured to be driven by a driver in one or more ways as described herein. Further, other features and/or functionalities not specifically described in reference to FIGS. 9 and 10 can be implemented in manners similar to those described in reference to FIGS. 4-6.

FIGS. 9A-9F show that in certain embodiments, the second body 652 can include a bone-engaging thread pattern dimensioned to engage the bone. In certain embodiments, the bone-engaging thread formed on the second body 652 can substantially similar in pitch and sectional shape as the bone-engaging thread formed on the first body 650 so that when the second body 652 is in its first position relative to the first body 650 (e.g., FIGS. 8A-8C), the second body's (652) thread substantially engages the bone via thread pattern formed in the bone by or for the first body's (650) thread. In certain embodiments, the second body's (652) bone-engaging thread substantially continues from the end of the bone-engaging thread of the first body 650. In certain embodiments, the thread pattern of the first and second bodies (650, 652) can be substantially continuous, even though the threads themselves may or may not be substantially continuous. For example, there may be a gap between the first body's thread and the second body's thread; however, the second body's thread can engage the bone via the thread pattern formed in the bone by or for the first body's thread.

Based on the foregoing example thread configuration for the first and second bodies (650, 652), the example suture anchor 640 can be driven into the bone such that the second body 652 does not significantly separate from the bone-engaging portion of the first body 650. FIG. 9A shows that in certain embodiments, a coupling interface 660 can be provided between the first and second bodies (650, 651). In certain embodiments, the coupling interface 660 can be configured to allow the second body 652 to follow the first body 650 into the bone without significant separation, until the second body 652 reaches a selected depth into the bone. As such a stage, the coupling interface 660 can be configured to allow the second body 652 to be separated from the bone-engaging portion of the first body 650 as the first body 650 is driven further into the bone. Non-limiting examples and design considerations for the coupling interface 660 are described below in greater detail.

In certain embodiments, the foregoing selected depth of the second body 652 at which the separation begins can be defined by a stop feature formed at a longitudinal location on the second body 652. In the example shown in FIGS. 9A-9F, such a stop feature can include a screw head-like stop structure 662 formed at a selected longitudinal location on the second body 652. The stop structure 662 can extend partially or substantially fully azimuthally along the outer surface of the second body 652. In certain embodiments, the leading side of the stop structure 662 can be dimensioned in a number of ways to inhibit the second body 652 from being driven into the bone when the applied torque on the suture anchor is less than some torque value.

In certain embodiments, the stop structure 662 can be dimensioned such that its overall diameter is less than the inner diameter of the suture retaining ring 654 so as to allow the retainer ring 654 to be constrained between the stop structure 662 and the flared portion 656 of the first body 656. Accordingly, the trailing side of the stop structure 662 can be dimensioned to facilitate locking of a suture, and to reduce the likelihood of damage to the suture during such a locking operation. For example, the trailing side and the outer portion of the stop structure 662 can be formed with smooth surfaces.

For the description of the example locking sequence depicted in FIGS. 9A-9F, it is assumed that the suture anchor 640 has been driven into the bone 104 such that the unseparated second body 652 is stopped from further insertion by the stop structure 662 (stage 700 in FIG. 9A). At such a stage, the stop structure 662 is depicted as being approximately at the bone surface level 106.

In FIG. 9A, the distance between the stop structure 662 and the flared portion 656 is indicated as D1, and the distance between the stop structure 662 and the leading end of the first body 650 is indicated as D2. It will be noted that D1+D2 represents a substantially constant overall length of the suture anchor 640.

In FIG. 9B, an example stage 702 shows that the second body 652 has begun separating from the bone-engaging portion of the first body 650. Accordingly, D1 decreases while D2 increases from those corresponding to the stage 700 of FIG. 9A.

In FIG. 9C, an example stage 704 shows that the second body 652 has separated sufficiently from the bone-engaging portion of the first body 650 so as to allow rotational disengagement between the first and second bodies (650, 652). At this example stage, D1 is less than, and D2 is greater than, those of the stage 702 of FIG. 9B.

In FIG. 9D, an example stage 706 shows that the first body 650 is being driven in further into the bone after being rotationally disengaged (FIG. 9C) from the second body 652. The second body 652 is substantially unable to be further driven into the bone due to the stop structure 662. Accordingly, the bone-engaging portion of the first body 650 moves further away from the second body 652 (D2 greater than that of FIG. 9C), and the flared portion 656 moves towards the stop structure 662 (D1 less than that of FIG. 9C).

In FIG. 9E, an example stage 708 shows that the first body 650 is being driven in further into the bone. At this example stage, D1 is less than, and D2 is greater than, those of the stage 706 of FIG. 9D.

In FIG. 9F, an example stage 710 shows that the first body 650 has been rotated relative to the second body 652 substantially fully, such that D1 is less than, and D2 is greater than, those of the stage 708 of FIG. 9E. At this stage, the stop structure 662, the suture retaining ring 654, and the flared portion 656 are dimensioned and spaced so as to provide a firm squeezing action for one or more sutures that is/are looped through the suture retaining ring. In certain embodiments, such dimensions and D1 spacing can be selected such that a substantially full suture lock can be achieved before the full rotational travel (e.g., 90 degrees) of the first body 750 relative to the second body 652. Such a feature can facilitate different-thickness sutures and/or sutures having different mechanical properties.

In the example shown in FIGS. 9A-9F, the inner surface of the second body 652 and the coupling section of the first body 650 are not threaded. In certain embodiments, the second body 652 can move substantially freely along the longitudinal direction from its first position (in engagement with the bone-engaging portion of the first body 650) to the suture lock position, if the suture anchor 640 is not embedded in a bone. In certain embodiments, it may be desirable to provide some friction between the first and second bodies 650, 652 so as to inhibit accidental or unwanted movement of the second body 652 from its first position prior to the separation stage as described in reference to FIG. 9A.

As shown, the coupling interface 660 can include an engaging surface 672 defined by an edge 670 at or near the trailing end of the bone-engaging portion of the first body 650. As also shown, an edge 680 at or near the leading end of the second body 652 includes an engaging surface 682. In certain embodiments, the edges 670 and 680 can be formed at an angle that is similar to the angle of the bone engaging thread.

In the example shown in FIGS. 9 and 10 (depicting a perspective view of the bone-engaging portion of the first body 650), the edge 670 can include first and second sections (670a, 670b) that are offset longitudinally by the engaging surface 672. In the particular example shown in FIG. 10, there are two sets of the edge/engaging surface combination disposed at substantially opposing sides. The edge 680 and engaging surface 682 combination(s) (not shown in FIG. 10) of the second body 652 can be dimensioned to substantially match with those for the bone-engaging portion of the first body 650.

In FIGS. 9 and 10, the engaging surfaces 672 and 682 are not necessarily depicted to scale. Also, although depicted for clarity in description, the engaging surfaces 672 and 682 may or may not be in the form of a step-like configuration. In some embodiments, the engaging surfaces 672 and 682 can be configured to provide a cam functionality or a cam-like coupling functionality, such that the rotational motion of the first body 650 results in a longitudinal movement of the first body's bone-engaging portion of the first body 650 away from the second body 652. Some design parameters that can be considered for the engaging surfaces are described below in greater detail.

In FIGS. 9 and 10, the two-set edge/engaging surface example configuration are dimensioned such that after the separation of the second body 652 from the bone-engaging portion of the first body 650, the first body 650 rotates approximately a quarter turn to achieve suture lock. Other configurations in the number of edge/engaging surface sets and/or the azimuthal displacement for suture lock can also be implemented.

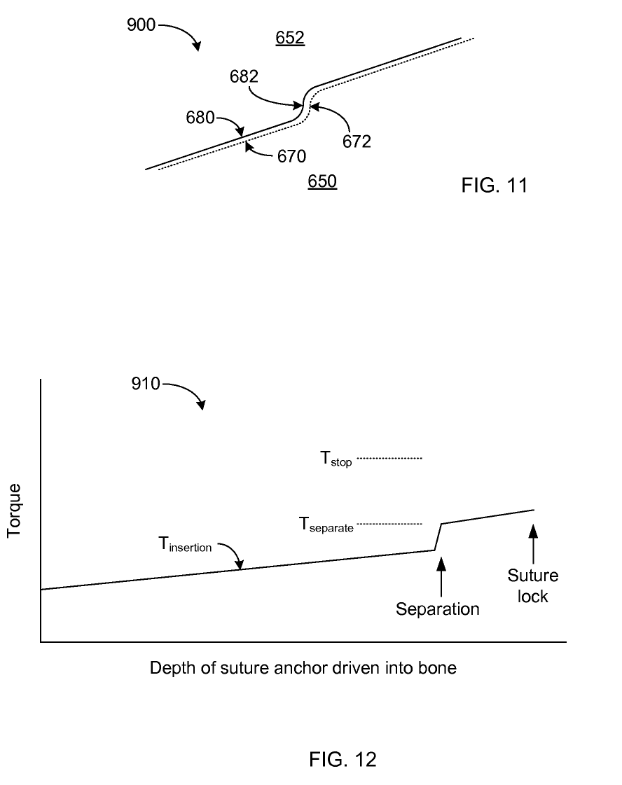

As described herein in reference to FIGS. 9 and 10, the engaging surfaces 672 and 682 of the first and second bodies (650, 652), respectively, can be configured in a number of ways. FIG. 11 shows an isolated view of an example interface 900 (similar to the interface 660 in FIG. 9A). In certain embodiments, one or more parameters associated with such an interface can be selected to provide a desired functionality of the interface 900. Pitch of the edges 670 and 680, length and angle of the engaging surfaces 672 and 682, and profile of the engaging surfaces 672 and 682 (e.g., sharp corners or rounded corners) are some non-limiting examples of such parameters.

In certain embodiments, one or more of the parameters associated with the interface between the first and second bodies (650, 652) can be selected based on one or more mechanical properties associated with driving of a suture anchor into a bone. FIG. 12 depicts an example torques curve 910 associated with such a process. As the suture anchor first engages the bone and is driven into the bone, the amount of torque needed to drive the anchor will likely increase as the anchor goes in deeper. A range of torque needed to drive the anchor from the bone surface to the second body separation stage (e.g., FIG. 9A) is generally depicted by a portion indicated as Tᵢₙₛₑᵣₜᵢₒₙ.

In certain embodiments, the interface between the first and second bodies (650, 652) can be configured so that the separation resulting in the rotational disengagement between the first and second bodies (650, 652) (e.g., FIG. 9C) is achieved by a torque Tₛₑₚₐᵣₐₜₑ that is greater than the torque applied arriving at the initial separation stage associated with FIG. 9A. Such a configuration can ensure that longitudinal separation does not occur prior to the second body 652 being embedded to a desired depth.

In certain embodiments, the stop structure (e.g., 662 in FIGS. 9A-9F) can be configured such that a torque Tₛₜₒₚ (that is greater than Tₛₑₚₐᵣₐₜₑ) is needed to drive the suture anchor beyond the stopped depth (e.g., the stop structure 662 at the surface 106). Such a configuration can ensure that the separating torque Tₛₑₚₐᵣₐₜₑ does not result in the suture anchor being undesirably driven further into the bone.

In the example shown in FIG. 12, further application of torque on the first body 650 (after the second body 652 is separated and rotationally disengaged from the bone-engaging portion of the first body 650) results in the second body 652 moving toward the suture lock position. In FIG. 12, such a torque is depicted as being greater than the separating torque and increasing therefrom. In certain embodiments, torque needed after the separation and rotational disengagement may not need to be to be greater than that of the separating torque.

In certain embodiments, a suture anchor having one or more features as described herein can be provided for use (e.g., surgical use) in an appropriate condition (e.g., in a substantially sterile package). In certain embodiments, a kit can include one or more of such suture anchors and one or more other devices (e.g., a driver and/or a suture). In certain embodiments, such a package or a kit can include an instruction for use that allows the user to implement one or more features or functionalities as described herein during the use of the suture anchor.

Conditional language, such as, among others terms, “can,” “could,” “might,” or “may,” and “preferably,” unless specifically stated otherwise, or otherwise understood within the context as used, is generally intended to convey that certain embodiments include, while other embodiments do not include, certain features, elements and/or steps.

Many variations and modifications can be made to the above-described embodiments, the elements of which are to be understood as being among other acceptable examples. Thus, the foregoing description is not intended to limit the scope of protection.

What is claimed is:

1. A suture anchor, comprising:
an elongate first body having first and second ends, the first body further comprising:
a bone-engaging section disposed adjacent the first end and having a first thread configured such that the bone-engaging section is capable of being driven into a bone;
a coupling section disposed between the bone-engaging section and the second end;
a second body disposed about the coupling section of the first body, the second body movable relative to the first body between a first position adjacent to the bone-engaging section and a second position that is closer to the second end of the first body, the second body further comprising a second thread configured such that when the second body is in the first position, at least a portion of the second thread is capable of engaging the bone by following the bone-engaging section when the bone-engaging section is driven into the bone;
a suture retaining member disposed between the second body and the second end of the first body, the suture retaining member capable of receiving a suture and configured such that when the second body moves to the second position, the suture is substantially secured relative to the suture retaining member;
a coupling mechanism formed on at least one of the first body and second body, the coupling mechanism configured to allow movement of the second body from the first position to the second position after at least a portion of the second thread engages the bone so as to facilitate the securing of the suture relative to the suture retaining member.

2. The suture anchor of claim 1, wherein the second end of the first body comprises a flared portion dimensioned to constrain the suture retaining member between the flared portion and the second body.

3. The suture anchor of claim 2, wherein the suture retaining member comprises a ring.

4. The suture anchor of claim 1, wherein the second body comprises an elongated collar that defines an interior surface dimensioned to substantially surround at least a portion of the coupling section.

5. The suture anchor of claim 4, wherein the coupling mechanism comprises a coupling thread formed on at least a portion of the coupling section and a matching coupling thread formed on at least a portion of the interior surface of the elongated collar, the coupling threads configured to allow the second body to move towards the second end of the first body when the first body is being driven into the bone and after the engagement of the second thread with the bone.

6. The suture anchor of claim 5, wherein the first and second threads of the first and second bodies and the matching coupling threads are configured such that the second end of the first body is approximately at the bone's surface when the second body reaches the second position to secure the suture.

7. The suture anchor of claim 4, wherein the coupling mechanism comprises a stop structure formed on an outer surface of the elongated collar, the stop structure configured to inhibit the elongated collar from driven further into the bone when the stop structure engages the bone's surface.

8. The suture anchor of claim 7, wherein the coupling mechanism further comprises a coupling interface between the first body and the second body, the coupling interface configured to force the second body to follow the bone-engaging section into the bone when the second body is in its first position.

9. The suture anchor of claim 8, wherein the coupling interface is further configured so that further application of driving torque after the stop structure's engagement with the bone's surface results in the elongated collar becoming rotationally disengaged from the bone-engaging section of the first body.

10. The suture anchor of claim 9, wherein the coupling interface comprises a cam surface defined on an end edge of the elongated collar and a substantially matching cam surface defined on an edge of the bone-engaging section.

11. The suture anchor of claim 10, wherein the cam surfaces are configured so as to provide a selected amount of longitudinal separation of the elongated collar followed by the rotational disengagement.

12. The suture anchor of claim 11, wherein the coupling section and the interior surface of the elongated collar have substantially smooth surfaces so as to facilitate both the longitudinal separation and rotational movement of the first body relative to the elongated collar as the first body is driven into the bone after the rotational disengagement.

13. The suture anchor of claim 11, wherein the stop structure is formed at the elongated collar's end towards the second end of the first body so as to allow the elongated collar to be substantially embedded in the bone before the rotational disengagement of the elongated collar from the bone-engaging portion of the first body.

14. The suture anchor of claim 13, wherein the elongated collar reaching its second position on the first body while being substantially embedded in the bone facilitates securing of the suture via engagement of the second thread of the elongated collar with the bone.

15. A kit, comprising:
the suture anchor of claim 1; and
a package for providing a desirable condition for the suture anchor.

16. The kit of claim 15, further comprising at least some instruction for use of the suture anchor.

17. The kit of claim 15, further comprising a driver configured to be capable of driving the suture anchor into a bone.

18. A method for securing a suture to a bone, the method comprising:
inserting a suture through a suture retaining ring that is part of an anchor, the anchor having a first member and a second member that is movably coupled to the first member, each of the first and second members having at least some bone-engaging features, the ring constrained between the first and second members and dimensioned to allow the inserting of the suture when the first and second members are in a first orientation and to secure the suture when the first and second members are in a second orientation;
attaching a driver to the anchor so as to allow turning of the anchor by providing torque to the driver;
turning the driver so as to drive the anchor into a bone such that the bone-engaging features of the first member engage with the bone;
turning the driver further to further drive the anchor such that the bone-engaging features of the second member engage with the bone;
sensing via the driver when the second member has been embedded in the bone at a selected depth;
providing an additional torque to the driver so as to induce movement of the first member relative to the second member; and
continuing to turn the driver until the first and second members reach the second orientation to thereby secure the anchor.
